(19) United States
(12) Patent Application Publication
(10) Pub. No.: US 2019/0217031 A1
(43) Pub. Date: Jul. 18, 2019

(54) Multi-Sensual Immersive Device And Systems And Methods Of Using Same

(71) Applicant: UNIVERSITY OF UTAH RESEARCH FOUNDATION, Salt Lake City, UT (US)

(72) Inventors: KAI KUCK, Park City, UT (US); Ben Chortkoff, Salt Lake City, UT (US)

(21) Appl. No.: 16/238,153

(22) Filed: Jan. 2, 2019

Related U.S. Application Data
(60) Provisional application No. 62/613,283, filed on Jan. 3, 2018.

(51) Int. Cl. A61M 16/00; A61M 16/06; A61M 21/00; A61M 16/08; A61M 16/10

(52) U.S. Cl. CPC A61M 16/022; A61M 16/06; A61M 16/0003; A61M 21/00; A61M 16/0875; A61M 2205/507; A61M 2021/0016; A61M 2021/005; A61M 2016/003; A61M 2205/3303; A61M 2205/3327; A61M 16/104

(57) ABSTRACT

A multi-sensual immersive system includes a face mask configured to cover a mouth and/or a nose of a patient and having an inlet configured to receive inhalational anesthetic. At least one breathing sensor is in fluid communication with the face mask and is configured to detect breathing of the patient. The system further includes a display and a computing device comprising. The computing device includes a processor and memory having instructions that, when executed, provide a game to a patient that is designed to manage the patient's breathing, facilitate a smooth induction of inhalational anesthesia, and reduce the patient's anxiety. The patient's breathing patterns can manipulate the game.

### CROSS-REFERENCE TO RELATED APPLICATIONS

This application claims priority to U.S. Provisional Application No. 62/613,283, filed Jan. 3, 2018, which is hereby incorporated by reference in its entirety (including its Appendices A and B) for all purposes.

### FIELD

This invention relates generally to systems for preparing a patient for anesthesia and, more specifically, to systems for directing the patient's breathing prior to and during anesthesia administration.

### BACKGROUND

Current methods of anesthesia induction can cause great anxiety, especially in pediatric patients. Anesthetic agents used to relieve this anxiety often last longer than the actual procedure and can place pediatric patients in a negative mental state. This can negatively impact preliminary recovery after the procedure and can result in prolonged hospital stays. In cases of multiple surgeries, traditional induction techniques can become increasingly difficult to perform. As such, an alternative non-pharmaceutical method for relieving patient anxiety is needed for the thousands of pediatric surgeries conducted each year.

### SUMMARY

Disclosed herein, in one aspect, is a multi-sensual immersive device and system.

A multi-sensual immersive system can include a face mask configured to cover a mouth and/or a nose of a patient and having an inlet configured to receive gas (e.g., a gas mixture including an inhalational anesthetic). At least one breathing sensor can be in fluid communication with the face mask and can be configured to detect breathing of the patient. The system can include a display and a computing device comprising a processor, and a memory device operatively coupled to the processor and having instructions thereon. The instructions, when executed, can perform a method comprising: displaying a game on the display, receiving outputs from the at least one breathing sensor indicative of breathing patterns of the patient, and providing feedback to the patient based on the breathing patterns by causing a change to the game on the display in response to a change in the breathing patterns.

The at least one breathing sensor can comprise at least one flow sensor (for example, a flow meter or a microphone).

Optionally, the at least one flow sensor can comprise both a flow meter and a microphone.

The computing device can be a smartphone or a mobile handheld device, and the display can be a screen of the smartphone or mobile handheld device.

The display can comprise a virtual reality display.

The memory device can have instructions thereon that, when executed, perform the method further comprising: filtering the outputs indicative of the breathing patterns of the patient to remove external noise.

The system can further comprise a camera, wherein the display is selectively changeable between a first mode that displays the game and a second mode that displays images captured by the camera in real time.

The system can further comprise a scent agent delivery unit, wherein the scent agent delivery unit is configured to release a scent agent into the face mask in response to a condition.

The memory device can have instructions thereon that, when executed, perform the method further comprising: displaying a visual scene corresponding with a known scent at a time during which inhalational anesthesia is being administered.

The system can further comprise a disposable wearable display headset.

A method can comprise detecting a breathing pattern of a patient, delivering an inhalational anesthetic to the patient through a face mask positioned over a mouth and/or a nose of the patient, and providing feedback on a display that instructs the patient to sequentially modify the breathing pattern to achieve a desired breathing pattern as the inhalational anesthetic is delivered.

The breathing pattern can be detected using a breathing sensor in fluid communication with the face mask.

Providing feedback on the display device can comprise providing feedback in the context of a game displayed on the display device as the patient participates in the game.

Providing feedback on the display device can comprise at least one of modifying at least one game performance indicator and providing the patient with a virtual item.

The game can be a virtual reality game.

The breathing sensor can comprise at least one flow sensor (for example, a flow meter or a microphone).

The breathing sensor can comprise a flow meter, and wherein detecting the breathing pattern of the patient comprises detecting a change in a flow rate from the flow meter.

The breathing sensor can comprise a microphone, and wherein detecting the breathing pattern of the patient comprises detecting a change in audio data from the microphone.

The method can further comprise delivering a scent agent to the face mask.

The method can further comprise displaying, on the display, a real time image captured on a camera that is coupled to the display.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a schematic of a multi-sensual immersive system in accordance with the present invention;

FIG. 2 is an image of a portion of the multi-sensual immersive system as in FIG. 1, showing a mask and a microphone;

FIG. 3 illustrates a perspective view of breathing sensors and their casing of the multi-sensual immersive system as in FIG. 1;

FIG. 4 illustrates a perspective view of a tube and adapter of the multi-sensual immersive system as in FIG. 1;

FIGS. 5A and 5B respectively illustrate raw audio breathing data and flow breathing data collected from the multi-sensual immersive system as in FIG. 1;

FIG. 6 illustrates filtered and overlaid breathing data of FIGS. 5A and 5B;

FIG. 7 illustrates elements of a game for use with the system as in FIG. 1;

FIG. 8 illustrates elements of another game for use with the system as in FIG. 1; and

FIG. 9 illustrates a computing device and operating environment for the multi-sensual immersive system as in FIG. 1.

### DETAILED DESCRIPTION

The present invention can be understood more readily by reference to the following detailed description and appendix, which include examples, drawings, and claims. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “a patient” can include two or more such patients unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, in the context of communication between multiple system components, the terms “coupled,” “operatively coupled,” “communicatively coupled,” and the like refer to any configuration that permits communication between the respective components in the manner disclosed herein. Thus, in the context of communication between multiple system components, these terms include, without limitation, wired connections, wireless connections, Internet-based connections (cable or WiFi), BLUETOOTH connections, radiofrequency transmission structures, telemetry-based structures, electrical connections, and the like.

Disclosed herein, in various aspects, are systems and methods for relieving anxiety in patients prior to and during induction of anesthesia.

Thus, disclosed herein are systems and methods for calming, distracting and engaging patients, particularly pediatric patients, to help relieve the anxiety associated with induction of anesthesia. In one aspect, disclosed is a game application that can utilize patient feedback in order to calm the patient and guide the patient through the induction procedure. In this aspect, the application can provide a calming immersive environment prior to and throughout the induction procedure by utilizing feedback from hardware accessories in order to advance through the game. At the end of induction, the game and all accessories can be easily removed so that clinicians can seamlessly transition to the procedure without contaminating the sterile environment.

In another aspect, the system disclosed herein can comprise an accessory to a mobile device, such as a smartphone, that can create an immersive, interactive experience for patients (e.g., pediatric patients and/or adults) undergoing anesthesia induction. In a further aspect, it is contemplated that the accessory can be disposable. In another aspect, the system can comprise a respiratory sensor and a patient face mask that can be configured to provide input to a computing device (e.g., a smartphone) that is executing an application (e.g., game) or movie, to incentivize a particular respiratory pattern conducive to improve and/or support anesthesia induction. In a further aspect, the immersive experience can include pleasant smells (e.g., flowers, strawberries) in the context of the application or movie, and can provide context (e.g., rocket launch, gas station) for the unpleasant smell of the inhalational anesthetic. In another aspect, it is contemplated that the accessory described herein can be sent home with the patient before the operation so that the patient can become familiar with the accessory and to provide the patient with educational content in preparation of the anesthesia and surgical experience. By accessing a camera of the computing device (e.g., a smartphone camera) or by design of the accessory, it is contemplated that the patient can choose to focus on the application or, instead, be aware of his or her surroundings. In another aspect, it is contemplated that the system can comprise other sensory experiences such as, for example and without limitation, an auditory experience.

It is contemplated that the devices, systems, and methods disclosed herein can provide a framework for providing non-threatening induction of anesthesia (or distract from the anesthesia induction) for anxious patients, thereby improving the patient experience. It is further contemplated that the devices, systems, and methods disclosed herein can replace the need for oral sedative medication, and can provide a feedback controlled incentive to breathe in a particular way to facilitate induction with an inhalational anesthetic agent. The hardware and the application can also provide opportunities to support hospital branding and advertisement, for example, by placement of labeling on the hardware or incorporating advertisements or other marketing messages into the application. It is contemplated that the educational component of the disclosed devices, systems, and methods can further improve the patient experience. In exemplary aspects, it is also contemplated that the disclosed systems can be used post-operatively in place of a disposable, costly incentive spirometer.

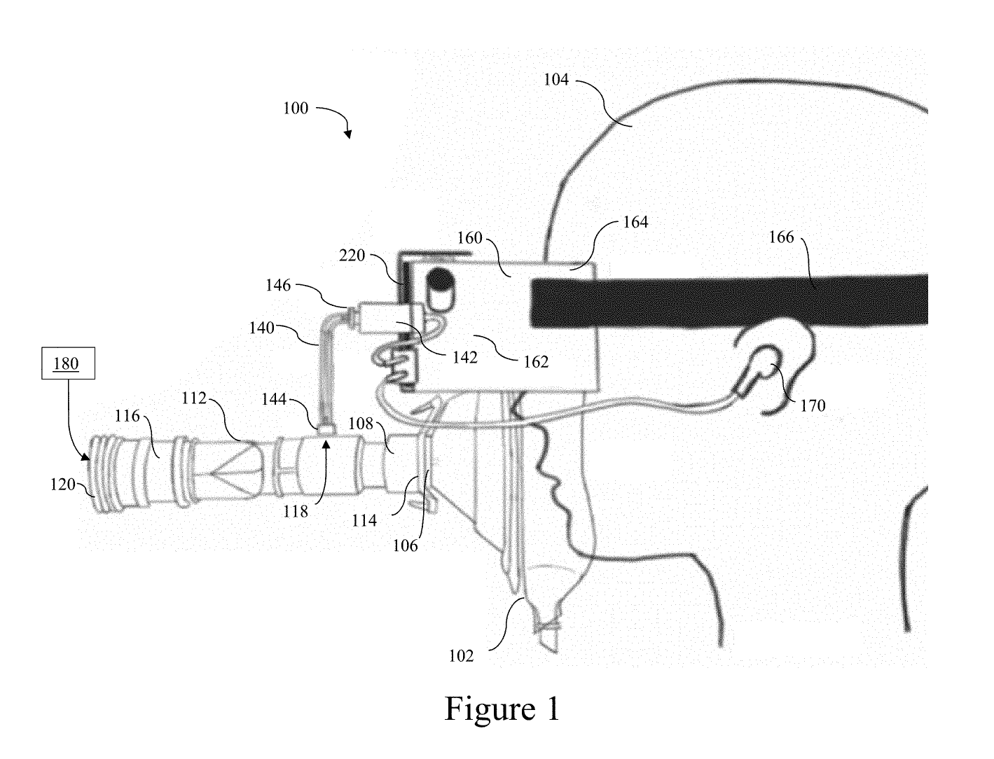

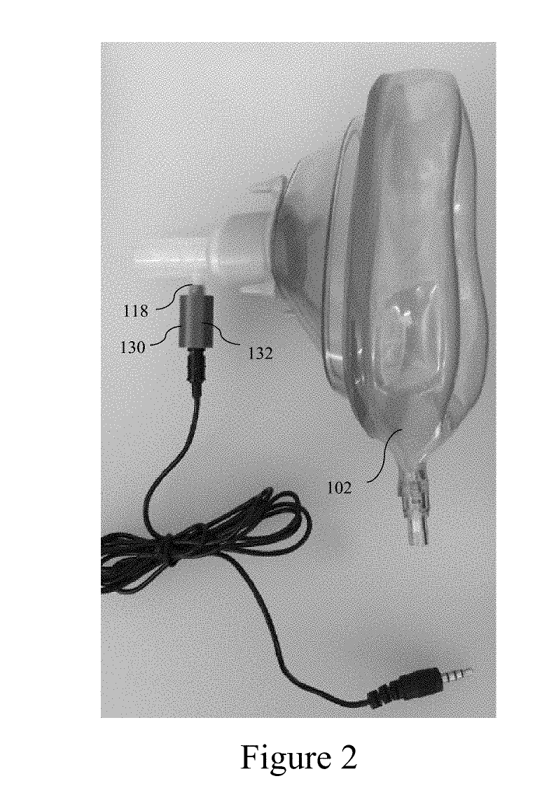

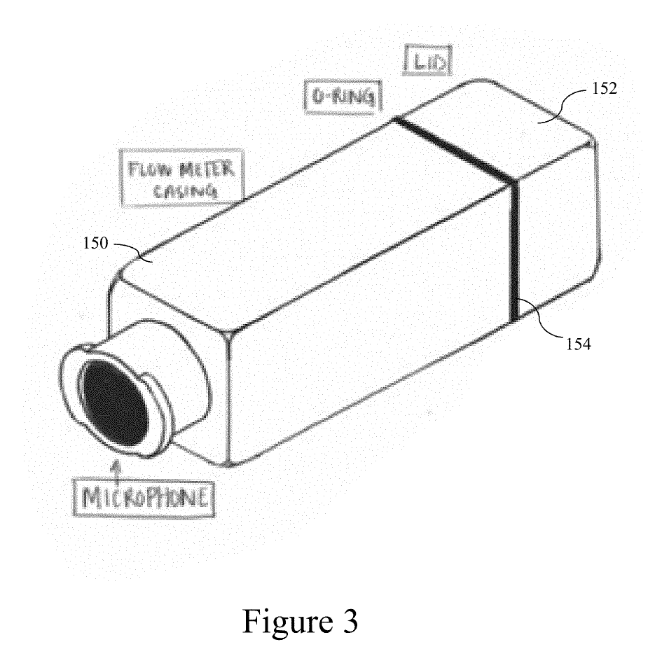

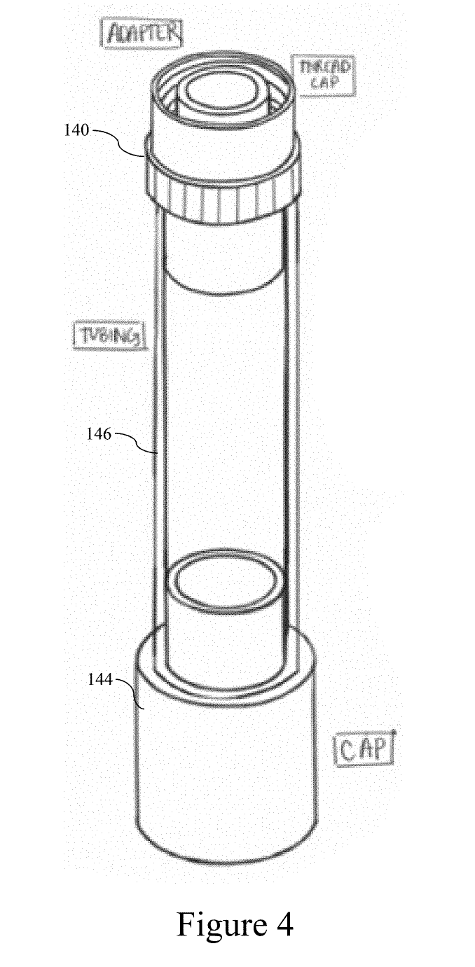

Referring to FIGS. 1-4, a multi-sensual system 100 can include an anesthesia face mask 102 that is configured to cover a mouth and/or a nose of a patient 104. The anesthesia face mask can include a main port 106 for receiving a main supply of air or gas (e.g., oxygen, nitrogen, combinations thereof, etc.) and inhalational anesthetic into the mask's internal volume as well as exhausting gas that the patient exhales. In some embodiments, the main port 106 can receive a gas mixture comprising one or more of oxygen, nitrous oxide, nitrogen, and inhalational anesthetic. The main port 106 can couple with an adapter 112 at the adapter's proximal end 114. The adapter 112 can couple at a distal end 116 with a hose 120 (shown in part) that provides for supply gas and exhalation exhaust. The adapter 112 can further include an outlet 118 that provides for communication with a microphone 130. In some embodiments, for example, as in FIG. 2, the outlet 118 can be a protrusion extending axially from the adapter 112 and defining a hole that allows for fluid communication between the adapter's interior and the outlet's distal end. The outlet's distal end can sealingly couple with a microphone adapter 132 that has the microphone 130 sealingly disposed therein so that minimal gas can leak between the adapter 112 and the microphone adapter 132 as well as between the microphone 130 and the microphone adapter 132. Additionally, or alternatively, in another embodiment, the outlet 118 can couple with a tube 140 that extends to a flow meter 142. The tube 140 can couple to the adapter 112 via a cap 144 at a first end. The tube 140 can couple to the flow meter 142 via a threaded cap 146 at a second end. The flow meter 142 can be disposed in a casing 150 that couples to a cap 152 and is sealed therebetween with an O-ring 154. In various embodiments, multi-sensual system 100 can comprise one or both of the microphone 130 and the flow meter 142. However, it is contemplated that any known flow sensor can be used. In some optional embodiments, the flow meter 142 can be an integral component of the adapter 112, as shown in U.S. Pat. No. 5,379,650 to Kofoed et al, which is hereby incorporated by reference in its entirety for all purposes. The flow meter 142 can be, for example, a differential pressure flow sensor. Moreover, the microphone 130 can be used to sense airflow.

Accordingly, the system 100 can include one or more flow sensors such as, for example, a microphone and/or a flow meter. For example, the flow sensor (e.g., the microphone 130 or the flow meter 142) can generate or collect data that is compared to a desired breathing pattern. In further optional aspects, both the microphone 130 and the flow meter 142 can collect various data that can be used to compare to a desired breathing pattern. The desired breathing pattern can include a desired breathing rate such as, for example, six to ten breaths per minute. Additionally, or alternatively, it is contemplated that the desired breathing pattern can include a specific quantity of air displaced during inhalation and/or exhalation, a specific flow rate, a particular shape or profile of a flow rate versus time curve during inhalation and exhalation, or combinations thereof.

The multi-sensual system 100 can further comprise a computing device and display, which can, according to some optional aspects, be collectively embodied as a smartphone 220. Although particularly described below as a smartphone, it is contemplated that other computing devices, such as tablets, portable computers, or PDA devices, can be used in the same or a similar manner. Thus, when the smartphone 220 is specifically described below, it should be understood that this disclosure encompasses the use of other computing devices in place of the smartphone 220. In one aspect, the smartphone 220 can be disposed within a wearable headset 160 that can allow the patient 104 to orient the smartphone 220 with respect to the patient's head and eyes. The smartphone 220 can further include one or more orientation/motion sensors (e.g., accelerometers, gyroscopes, and the like.) that can sense the smartphone's orientation and/or movement. In this way, the smartphone 220 can operate as a virtual reality device that integrates the motion and orientation of the smartphone with the game or movie that is displayed by the smartphone. The headset 160 can include a mount 162 that spaces the smartphone 220 a select (optionally, adjustable) distance from the patient's eyes. The mount 162 can include covers 164 that block ambient light, thereby allowing the wearer to focus on the display and ignore the surrounding environment. The mount can couple with a strap 166 that can secure the wearable headset to the patient's head. The strap can optionally be elastic and/or have an adjustable length to permit use by patients of varying sizes. Optionally, the strap can selectively be secured via hook and loop fastener. The headset 160 can further include lenses disposed therein. In some optional embodiments, the headset can be a GOOGLE CARDBOARD viewer and can be disposable after each use.

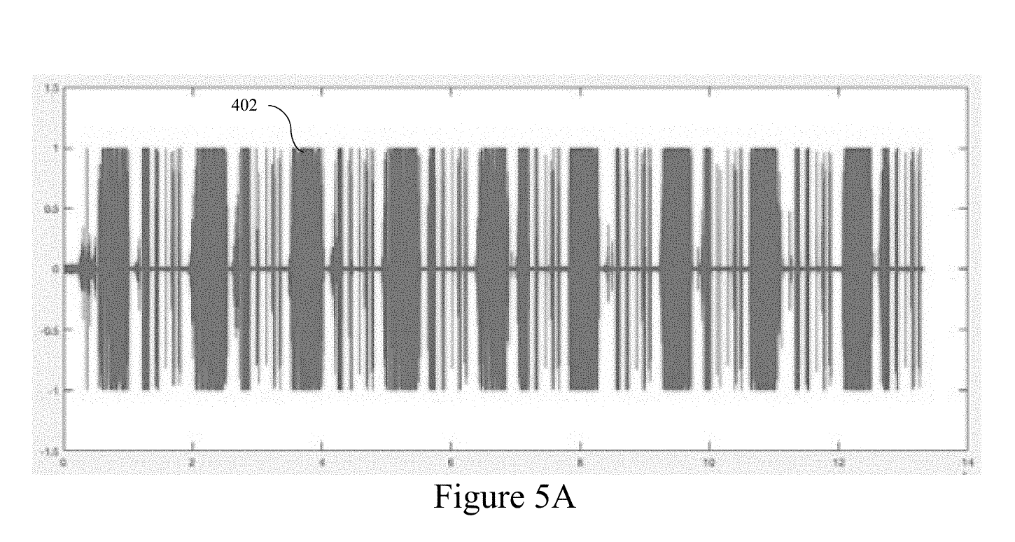

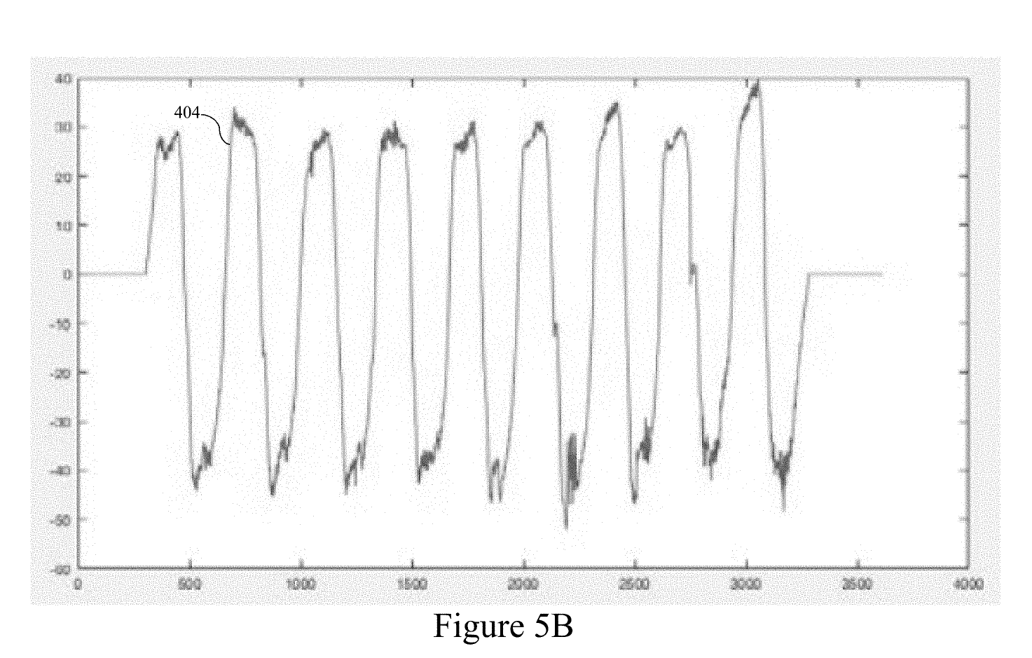

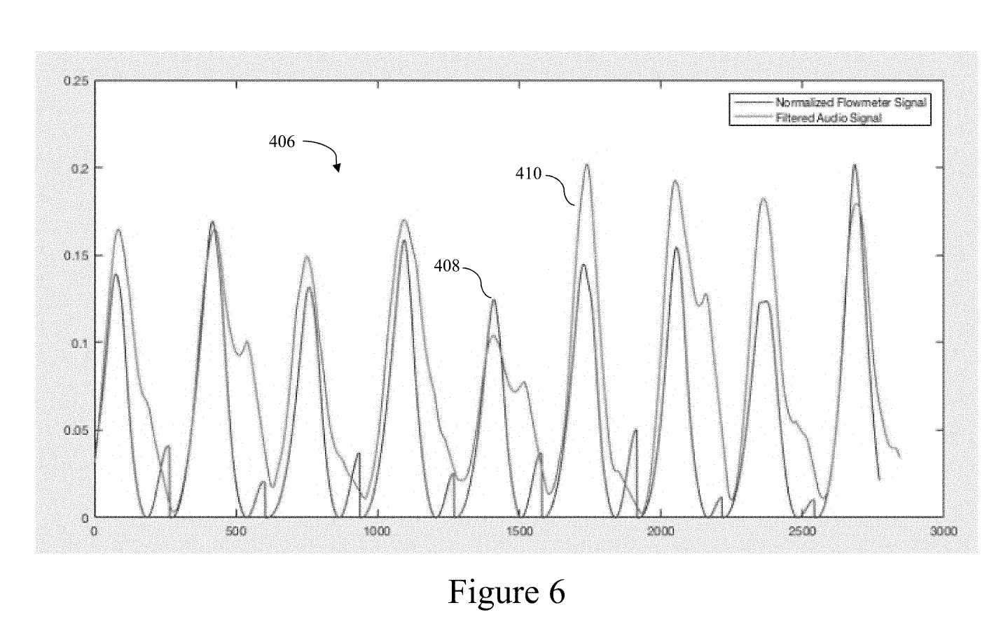

The microphone 130 and flow meter 142 can independently or collectively act as breathing sensors. Thus, it is contemplated that the microphone 130 can be used without the flow meter 142 (with the flow meter being omitted), and it is further contemplated that the flow meter 142 can be used without the microphone 130 (with the microphone being omitted). Referring to FIGS. 5A, 5B, and 6, the breathing sensors can capture raw breathing data such as, for example, audio data 402 (from the microphone 130) and flow data 404 (from the flow meter). The computing device can receive the raw breathing data from the breathing sensors. Next, the computing device can perform initial computations on the data. For example, the computing device 201 can filter the breathing data to remove noise using, for example, a high pass or low pass filter. Further, the computing device can apply a moving average to the breathing data. Additionally, the computing device can truncate and align the breathing signals. Still further, the computing device can normalize the breathing data. In further embodiments, the computing device can apply more than one of the above filters as well as other filtering methods known in the art but not explicitly described. In this way, the computing device can produce filtered breathing data 406 that can comprise filtered audio data 408 and filtered flow data 410. Optionally, in use, it is contemplated that the microphone 130 can detect intensities of 10 dB+/−5 dB or of 5 dB+/−2.5 dB.

The breathing data, either filtered or raw, can be compared to a desired breathing signal (corresponding to the desired breathing pattern). For example, a frequency of breaths, an amplitude of breaths, a volume of breaths, a consistency between breaths, individual breath patterns, or combinations thereof can be compared to the desired breathing signal.

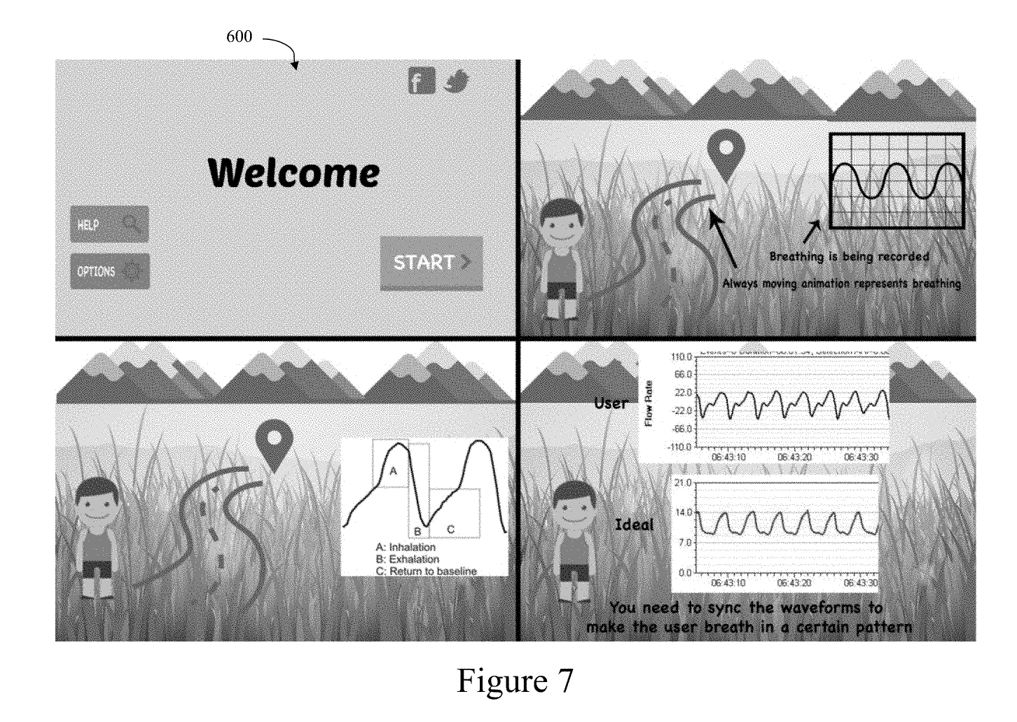

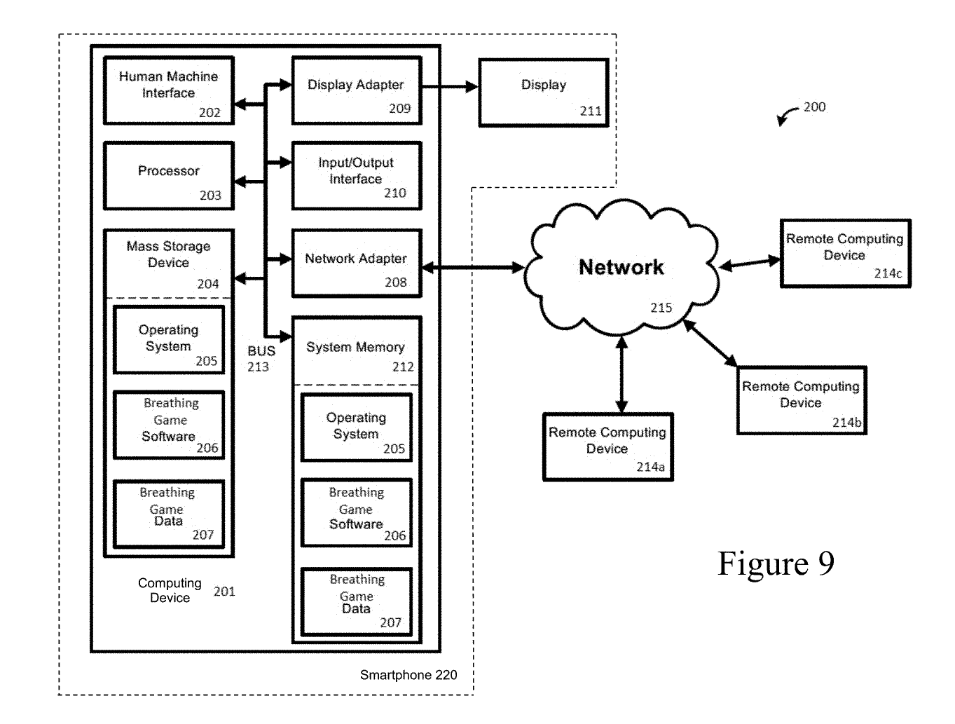

Referring to FIGS. 7 and 9, the computing device 201 can have in its memory instructions that when executed, enable the patient to play a game 600. The game can include or incorporate breathing exercises that encourage a desired type of breathing. For example, the patient can be prompted to take deep breaths in order to score points and/or advance within the game environment. According to various aspects, the game can prompt the patient to inhale at certain intervals and for certain durations as well as to exhale at certain intervals and for certain durations. As the patient breathes, the breathing sensors (e.g., the microphone 140 and flow sensor 142) can capture the patient's breathing patterns. Based on the patient's breathing patterns, the game can provide feedback to the patient. For example, if the patient does not breathe deeply enough, the game can prompt the patient accordingly and encourage the patient to continue trying. If, on the other hand, the patient breathes as prompted, the patient can score points, advance levels, obtain power ups or virtual items, etc. In this context, it is understood that the feedback provided by the game corresponds to visual representations, commands, and/or text shown on the display of the smartphone 220. For example, the visual scenes or text depicted on the display can be modified to encourage the patient to breath in a particular way (e.g., breathe in, breathe out, duration of inhale, duration of exhale, and the like). Optionally, the visual scenes depicted on the display can include a virtual representation of the patient (i.e., a character in the game) such that the patient can observe the motion, activity, and/or behavior of the character and then provide appropriate breathing actions to achieve a desired modification to the motion, activity, and/or behavior of the character within the game. Accordingly, the game can encourage the patient to breathe as desired, which can help to relax the patient as well as optimize inhalational anesthetic induction. At the same time, the game can distract the patient from surrounding events that can cause anxiety.

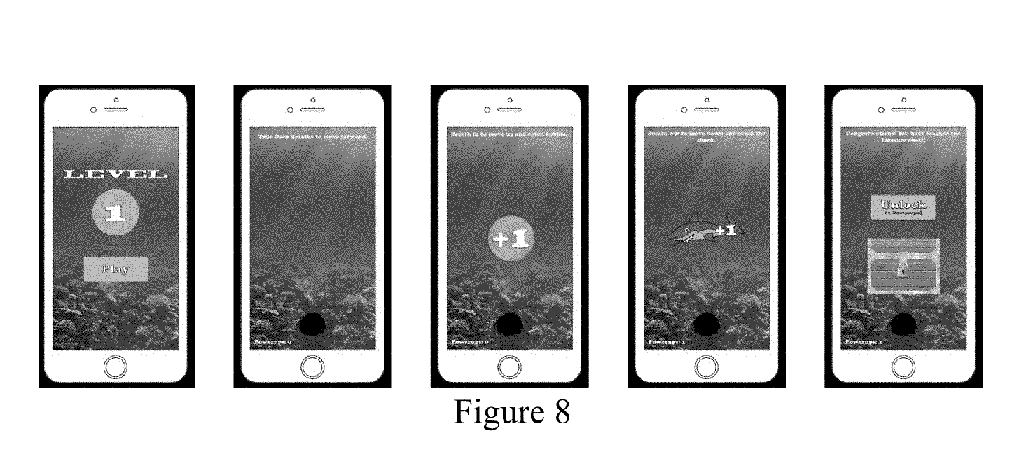

Referring to FIG. 8, in one embodiment, the game can be an underwater swimming game. The patient can be prompted to breathe deeply. The patient can then virtually advance forward by breathing deeply. The patient can next be prompted to breathe in to catch bubbles. The bubbles can be shown on the display at specific times to encourage the patient to breathe at specific intervals. The patient can further be prompted to exhale to swim away from a virtual shark. The patient can avoid the virtual shark by exhaling at desired times or for desired durations. If the patient sufficiently breathes as prompted, the patient can achieve certain goals and be virtually rewarded.

Referring to FIGS. 1 and 9, in optional aspects, scent agents can be delivered into the face mask 102 upon certain conditions. For example, a scent agent delivery unit 180 can deliver pleasant smells (e.g., flowers, strawberries) to the patient in the context of the game or a movie. It is contemplated that the scent agent delivery unit 180 can store one or more scent agents and be in fluid communication with the face mask 102. Optionally, the computing device 201 can be communicatively coupled to the scent agent delivery unit 180 (optionally, through a valve or actuator as is known in the art) to permit selective delivery of the scent agent upon predetermined conditions (e.g., triggering events or measurements). In further situations, the computing device 201 and the display 211 can, during administration of the inhalational anesthetic, provide a context (e.g., rocket launch, gas station) in which the unpleasant smell of the inhalational anesthetic would seem consistent and, thus, less noticeable or anxiety-inducing.

The multi-sensual system 100 can further include headphones 170 or other speakers that couple with the computing device and provide audio consistent with the game, movie, application, and the like. Thus, in some aspects, it is contemplated that feedback to the patient can be provided using sounds or audible commands that are delivered through the headphones or speakers.

FIG. 9 shows an exemplary computing system 200 that can be used with system 100. Computing system 200 can include a computing device 201 and a display 211 in electronic communication with the computing device. In some optional embodiments, and as shown in FIG. 9, a smartphone 220 can comprise both the computing device 201 and the display 211. Alternatively, it is contemplated that the display 211 can be provided as a separate component from the computing device 201. For example, it is contemplated that the display 211 can be in wireless communication with the computing device 201, thereby allowing usage of the display 211 in a manner consistent with that of the display of the smartphone as disclosed herein.

The computing device 201 may comprise one or more processors 203, a system memory 212, and a bus 213 that couples various components of the computing device 201 including the one or more processors 203 to the system memory 212. In the case of multiple processors 203, the computing device 201 may utilize parallel computing.

The bus 213 may comprise one or more of several possible types of bus structures, such as a memory bus, memory controller, a peripheral bus, an accelerated graphics port, and a processor or local bus using any of a variety of bus architectures.

The computing device 201 may operate on and/or comprise a variety of computer readable media (e.g., non-transitory). Computer readable media may be any available media that is accessible by the computing device 201 and comprises, non-transitory, volatile and/or non-volatile media, removable and non-removable media. The system memory 212 has computer readable media in the form of volatile memory, such as random access memory (RAM), and/or non-volatile memory, such as read only memory (ROM). The system memory 212 may store data such as breathing data 207 and/or program modules such as operating system 205 and breathing game software 206 that are accessible to and/or are operated on by the one or more processors 203.

The computing device 201 may also comprise other removable/non-removable, volatile/non-volatile computer storage media. A mass storage device 204 may provide non-volatile storage of computer code, computer readable instructions, data structures, program modules, and other data for the computing device 201. The mass storage device 204 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 204. An operating system 205 and the breathing game software 206 may be stored on the mass storage device 204. One or more of the operating system 205 and the breathing game software 206 (or some combination thereof) may comprise program modules and the breathing game software 206. Breathing game data 207 may also be stored on the mass storage device 204. The breathing game data 207 may be stored in any of one or more databases known in the art. The databases may be centralized or distributed across multiple locations within the network 215.

A patient may enter commands and information into the computing device 201 via an input device (not shown). Such input devices comprise, but are not limited to, a keyboard, a pointing device (e.g., a computer mouse, remote control), a microphone, a joystick, a scanner, tactile input devices such as gloves, and other body coverings, motion sensor, and the like. These and other input devices may be connected to the one or more processors 203 via a human machine interface 202 that is coupled to the bus 213, but may be connected by other interface and bus structures, such as a parallel port, game port, an IEEE 1394 Port (also known as a Firewire port), a serial port, network adapter 208, and/or a universal serial bus (USB).

A display 211 may also be connected to the bus 213 via an interface, such as a display adapter 209. It is contemplated that the computing device 201 may have more than one display adapter 209 and the computing device 201 may have more than one display 211. A display 211 may be a monitor, an LCD (Liquid Crystal Display), light emitting diode (LED) display, television, smart lens, smart glass, and/or a projector. In addition to the display 211, other output peripheral devices may comprise components such as speakers (not shown) and a printer (not shown) which may be connected to the computing device 201 via Input/Output Interface 210. Any step and/or result of the methods may be output (or caused to be output) in any form to an output device. Such output may be any form of visual representation, including, but not limited to, textual, graphical, animation, audio, tactile, and the like. The display 211 and computing device 201 may be part of one device, or separate devices.

The computing device 201 may operate in a networked environment using logical connections to one or more remote computing devices 214a,b,c. A remote computing device 214a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 201 and a remote computing device 214a,b,c may be made via a network 215, such as a local area network (LAN) and/or a general wide area network (WAN). Such network connections may be through a network adapter 208. A network adapter 208 may be implemented in both wired and wireless environments. Such networking environments are conventional and commonplace in dwellings, offices, enterprise-wide computer networks, intranets, and the Internet. In further exemplary aspects, it is contemplated that the computing device 201 can be in communication with the remote computing devices 214a,b,c, e.g., through a Cloud-based network.

Application programs and other executable program components such as the operating system 205 are shown herein as discrete blocks, although it is recognized that such programs and components may reside at various times in different storage components of the computing device 201, and are executed by the one or more processors 203 of the computing device 201. An implementation of the breathing game software 206 may be stored on or sent across some form of computer readable media. Any of the disclosed methods may be performed by processor-executable instructions embodied on computer readable media.

In some embodiments, the remote computing device 214a can provide an interface through which a clinician can control aspects of the game as the game is executed through the computing device 201. Through the interface of the remote computing device 214a, the clinician may be able to trigger the computing device 201 to show scenes on the display 211 that correspond to the scent associated with the inhalational anesthetic. In further embodiments, certain triggers in the game/application can be automatic. For example, upon receiving information from an anesthesia machine that the inhalational anesthetic is being delivered, the computing device 201 can be automatically triggered to show scenes on the display 211 that correspond to the scent associated with the inhalational anesthetic. Thus, in these aspects, it is contemplated that the computing device 201 can be communicatively coupled to a processing unit of the anesthesia machine to receive an indication of delivery of the inhalational anesthetic. In exemplary aspects, the interface of the remote computing device 214a can comprise a graphical user interface. Additionally, or alternatively, the interface of the remote computing device 214a can comprise an input device, such as for example and without limitation, a keyboard, joystick, or touchpad that is communicatively coupled to the remote computing device to provide a mechanism for the clinician to selectively deliver an instructional input.

The game can be designed for a patient of a given age. For example, various games that are appropriate for patients at a variety of different ages can be implemented on the system 100. The game may be selectively paused (either manually or automatically, for example, when the smartphone 220 is removed from the patient's head). In some embodiments, it is contemplated that the clinician can have the option to pause the game from the remote computing device 214a. The pausing feature may be advantageous, for example, in situations in which the patient is being transported within a hospital or other clinical setting.

In exemplary aspects, it is contemplated that the computing device 201 can comprise an existing gaming device that is configured to execute a pre-existing game that is specifically designed for use with the gaming device. In these aspects, it is contemplated that the pre-existing game can be adapted to interface with the breathing of the user using the face mask and flow sensors as disclosed herein. That is, the system 100 can be adapted to serve as a substitute or replacement for standardized game controllers, which typically include buttons, switches, and/or joysticks that can be selectively engaged or manipulated to control actions within the game environment (without the need for determining or measuring breathing parameters). In exemplary aspects, it is contemplated that the flow sensors of the system 100 can detect breathing in the manner disclosed herein, with the breathing data being used to control actions within the environment of the pre-existing game. In these aspects, it is contemplated that the system 100 can include an electronic communication link (e.g., a cable or wireless transmission structure) that provides communication between the existing gaming device and the flow sensors to permit responsive control during game play.

Although several embodiments of the invention have been disclosed in the foregoing specification and the following appendices, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed herein, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system comprising: a face mask configured to cover at least one of a mouth and a nose of a patient and having an inlet configured to receive inhalational anesthetic; at least one breathing sensor in fluid communication with the face mask and that is configured to detect breathing of the patient; a display; and a computing device comprising: a processor, and a memory device operatively coupled to the processor and having instructions thereon that, when executed perform a method comprising: displaying a game on the display, receiving outputs from the at least one breathing sensor indicative of breathing patterns of the patient, and providing feedback to the patient based on the breathing patterns by causing a change to the game on the display in response to a change in the breathing patterns.

Aspect 2: The system of aspect 1, wherein the at least one breathing sensor comprises a flow meter or a microphone.

Aspect 3: The system of aspect 1, wherein the at least one breathing sensor comprises a flow meter and a microphone.

Aspect 4: The system of aspect 1, wherein the computing device is a smartphone, and the display is a screen of the smartphone.

Aspect 5: The system of aspect 1, wherein the display comprises a virtual reality display.

Aspect 6: The system of aspect 1, wherein the memory device has instructions thereon that, when executed, perform the method further comprising: filtering the outputs indicative of the breathing patterns of the patient to remove external noise.

Aspect 7: The system of aspect 1, further comprising a camera, wherein the display is selectively changeable between a first mode that displays the game and a second mode that displays images captured by the camera in real time.

Aspect 8: The system of aspect 1, further comprising a scent agent delivery unit, wherein the scent agent delivery unit is configured to release a scent agent into the face mask in response to a condition.

Aspect 9: The system of aspect 1, wherein the memory device has instructions thereon that, when executed, perform the method further comprising: displaying a visual scene corresponding with a known scent at a time during which inhalational anesthesia is being administered.

Aspect 10: The system of aspect 1, further comprising a disposable wearable display headset.

Aspect 11: A method comprising: detecting a breathing pattern of a patient; delivering a gas mixture comprising an inhalational anesthetic to the patient through a face mask positioned over at least one of a mouth and a nose of the patient; and providing feedback on a display that instructs the patient to sequentially modify the breathing pattern to achieve a desired breathing pattern as the inhalational anesthetic is delivered.

Aspect 12: The method of aspect 11, wherein the breathing pattern is detected using a breathing sensor in fluid communication with the face mask.

Aspect 13: The method of aspect 12, wherein providing feedback on the display device comprises providing feedback in the context of a game displayed on the display device as the patient participates in the game.

Aspect 14: The method of aspect 13, wherein providing feedback on the display device comprises at least one of modifying at least one game performance indicator and providing the patient with a virtual item.

Aspect 15: The method of aspect 13, wherein the game is a virtual reality game.

Aspect 16: The method of aspect 12, wherein the breathing sensor comprises at least one of a flow meter and a microphone.

Aspect 17: The method of aspect 16, wherein the breathing sensor comprises a flow meter, and wherein detecting the breathing pattern of the patient comprises detecting a change in a flow rate from the flow meter.

Aspect 18: The method of claim 16, wherein the breathing sensor comprises a flow meter, and wherein detecting the breathing pattern of the patient comprises detecting a change in a pressure from the flow meter.

Aspect 19: The method of any one of aspects 16-18, wherein the breathing sensor comprises a microphone, and wherein detecting the breathing pattern of the patient comprises detecting a change in audio data from the microphone.

Aspect 20: The method of any one of aspects 12-19, further comprising delivering a scent agent to the face mask.

Aspect 21: The method of any one of aspects 12-20, further comprising displaying, on the display, a real time image captured on a camera that is coupled to the display.

What is claimed is:

1. A system comprising:
a face mask configured to cover at least one of a mouth and a nose of a patient and having an inlet configured to receive a gas mixture comprising inhalational anesthetic;
at least one breathing sensor in fluid communication with the face mask and that is configured to detect breathing of the patient;
a display; and
a computing device comprising:
a processor, and
a memory device operatively coupled to the processor and having instructions thereon that, when executed perform a method comprising:
displaying a game on the display,
receiving outputs from the at least one breathing sensor indicative of breathing patterns of the patient, and
providing feedback to the patient based on the breathing patterns by causing a change to the game on the display in response to a change in the breathing patterns.

2. The system of claim 1, wherein the at least one breathing sensor comprises a flow meter or a microphone.

3. The system of claim 1, wherein the at least one breathing sensor comprises a flow meter and a microphone.

4. The system of claim 1, wherein the computing device is a smartphone, and the display is a screen of the smartphone.

5. The system of claim 1, wherein the display comprises a virtual reality display.

6. The system of claim 1, wherein the memory device has instructions thereon that, when executed, perform the method further comprising: filtering the outputs indicative of the breathing patterns of the patient to remove external noise.

7. The system of claim 1, further comprising a camera, wherein the display is selectively changeable between a first mode that displays the game and a second mode that displays images captured by the camera in real time.

8. The system of claim 1, further comprising a scent agent delivery unit, wherein the scent agent delivery unit is configured to release a scent agent into the face mask in response to a condition.

9. The system of claim 1, wherein the memory device has instructions thereon that, when executed, perform the method further comprising: displaying a visual scene corresponding with a known scent at a time during which inhalational anesthesia is being administered.

10. The system of claim 1, further comprising a disposable wearable display headset.

11. A method comprising:
detecting a breathing pattern of a patient;
delivering a gas mixture comprising an inhalational anesthetic to the patient through a face mask positioned over at least one of a mouth and a nose of the patient; and
providing feedback on a display that instructs the patient to sequentially modify the breathing pattern to achieve a desired breathing pattern as the inhalational anesthetic is delivered.

12. The method of claim 11, wherein the breathing pattern is detected using a breathing sensor in fluid communication with the face mask.

13. The method of claim 12, wherein providing feedback on the display device comprises providing feedback in the context of a game displayed on the display device as the patient participates in the game.

14. The method of claim 13, wherein providing feedback on the display device comprises at least one of modifying at least one game performance indicator and providing the patient with a virtual item.

15. The method of claim 13, wherein the game is a virtual reality game.

16. The method of claim 12, wherein the breathing sensor comprises a flow meter or a microphone.

17. The method of claim 16, wherein the breathing sensor comprises a flow meter, and wherein detecting the breathing pattern of the patient comprises detecting a change in a flow rate from the flow meter.

18. The method of claim 16, wherein the breathing sensor comprises a flow meter, and wherein detecting the breathing pattern of the patient comprises detecting a change in a pressure from the flow meter.

19. The method of claim 16, wherein the breathing sensor comprises a microphone, and wherein detecting the breathing pattern of the patient comprises detecting a change in audio data from the microphone.

20. The method of claim 12, further comprising delivering a scent agent to the face mask.

21. The method of claim 12, further comprising displaying, on the display, a real time image captured on a camera that is coupled to the display.
